(19) United States
(12) Patent Application Publication
(10) Pub. No.: US 2021/0375395 A1
(43) Pub. Date: Dec. 2, 2021

(54) Omics Detection of Nonhomologous End Joining Repair Site Signatures

(71) Applicant: Nantomics, Culver City, CA (US)

(72) Inventor: Shahrooz Rabizadeh, Agoura Hills, CA (US)

(21) Appl. No.: 17/287,956

(22) PCT Filed: Dec. 5, 2019
(86) PCT No.: PCT/US2019/064680

§ 371 Date: Apr. 22, 2021

Related U.S. Application Data
(60) Provisional application No. 62/776,060, filed on Dec. 6, 2018.

(51) Int. Cl. G16B 25/10; G16B 5/20; G16B 20/00; G16B 30/00; G06N 5/04; G16H 40/63

(52) U.S. Cl. CPC G16B 25/10; G16B 5/20; G16H 40/63; G16B 30/00; G06N 5/04; G16B 20/00

(57) ABSTRACT

A molecular signature of an error-prone DNA repair can be identified from an analysis of omics data set obtained from a tumor tissue or a patient having a tumor. The identified molecular signature can be associated with a causation, a prognosis, or a treatment option of the tumor, and further used to determine a treatment regimen effective to treat the tumor.

This application claims priority to co-pending U.S. Provisional Application No. 62/776,060, filed on Dec. 6, 2018, the entire content of which is herein incorporated by reference.

### FIELD OF THE INVENTION

The field of the invention is computational analysis of omics data, and particularly as it relates to identification of mutational signature of non-homologous end joining repair site.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Non-homologous end joining (NHEJ) is a pathway that repairs double-strand breaks in DNA, which uses short homologous DNA sequences (microhomologies) to initiate the repair process. The NHEJ is generally considered an error-prone DNA-repair process due to its relatively high chance of causing a mutation in a gene by adding, deleting, or substituting several nucleotides at the end of the double-strand breaks when the process is not accurate or the surveillance of the double-strand breaks is inactivated or attenuated. Such erroneously added or deleted nucleotides at the end of the double-strand breaks may further cause chromosomal translocation or even structural change (e.g., fusion of two chromosomes, etc.), which can be often observed in the tumor cells.

Several factors have been identified to attribute to the inefficient or inaccurate NHEJ pathway, and further identified to be associated with one or more types of cancer. For example, inactivation or absence of core elements of the NHEJ process including Ku70/80, DNA-PKcs and XRCC4/LigIV may lead to reduction in the NHEJ efficiency and/or fidelity to cause insertion and/or deletion at the junction of the double-strand breaks. In addition, inactivation of DNA damage surveillance or response complex including BRCA1 or BRCA2 may reduce the efficiency and selectivity of the DNA repair pathways (including homologous recombination pathway and NHEJ that can be competitive with each other). Consequently, several studies have indicated that inhibition or reduction of the DNA damage surveillance or response complex may lead to various types of cancer, including, most notoriously, a breast cancer, by proceeding with more error-prone NHEJ pathway rather than homologous recombination pathway. In addition, inhibition of NHEJ pathway may even lead to impairment of tumor growth (e.g., pancreatic cancer). Li at al., Plos One, Vol. 7, Issue 6, e39588 (June 2012).

Therefore, even if relations between error-prone DNA repair pathways and tumor prognosis are somewhat known in the art, it is largely unexplored how to identify specific tumors related to the error-prone DNA repair pathways using omics data of a patient and further to develop a treatment plan for such tumors. Thus, there is still a need for improved systems and methods for analyzing omics data of a patient to identifying a molecular signature of the error-prone DNA repair in the tumor.

### SUMMARY OF THE INVENTION

The inventive subject matter is directed to various methods for analyzing omics data of a patient for identifying a molecular signature of the error-prone DNA repair in the tumor. Thus, in one inventive subject matter, the inventors contemplate a method of analyzing omics data of a patient having a tumor. In this method, omics data sets of the tumor from the patient are obtained, and a molecular signature of an error-prone DNA repair in the tumor is identified from the omics data sets of the tumor. Then, the molecular signature is associated with at least one of a causation, a prognosis, and a treatment option of the tumor. In some embodiments, the causation comprises a mutation in at least one of BRCA1 and BRCA2.

Most typically, the omics data sets include at least two selected from genomics data, transcriptomics data, and proteomics data. Preferably, the genomics data comprises a whole genome sequencing data, a whole exome sequencing data or a copy number data. Also preferably, the transcriptomics data comprises RNA sequencing data, RNA expression level data, or allele fraction data. In some embodiments, the omics data sets include genomics data of a circulating tumor DNA or a transcriptomics data of a circulating tumor RNA.

In some embodiments, the error-prone DNA repair is a non-homologous end repair joining, and/or the molecular signature comprises at least one of insertion or deletion of a nucleic acid fragment in a genome, wherein a size of the nucleic acid fragment is between 25-50 base pairs. Thus, in some embodiments, the step of identifying the molecular signature comprises comparing a genome sequencing data of the tumor with a genome sequencing data of a matched normal tissue.

Additionally, the method may further comprise steps of obtaining a pathway model comprising a plurality of pathway elements and a plurality of regulatory parameters and inferring an activity of a tumor-associated protein using the pathway model and the omics data sets. Most preferably, at least one of the pathway elements and the regulatory parameters includes the molecular signature of the error-prone DNA repair. In such embodiments, it is also preferred that the method further comprises modulating the pathway model based on the inferred activity of the tumor-associated protein, and/or determining a treatment regimen to include a treatment targeting the tumor-associated protein.

In some embodiments, the method may further comprise a step of determining an RNA expression level of a portion of a genome having the molecular signature. Then, preferably, the method may further continue with a step of determining a treatment regimen to include a treatment targeting the portion of the genome.

In some embodiments, the method may further comprise a step of determining a treatment regimen based on the at least one of the causation and the prognosis of the tumor. For example, the causation is a mutation in at least one of BRCA1 and BRCA2, and the treatment regimen is a PARP inhibitor.

In another aspect of the inventive subject matter, the inventors contemplate a method of predicting effectiveness of a PARP inhibitor in treating a tumor of a patient. In this method, genomics data and transcriptomics data of the tumor from the patient is obtained. A molecular signature of an error-prone DNA repair in the tumor is identified from the genomics data of the tumor, and an expression level of a portion of a genome having the molecular signature is determined using the transcriptomics data. Then, the effectiveness of a PARP inhibitor can be predicted based on the molecular signature and the expression level. In some embodiments, the effectiveness of the PARP inhibitor is predicted high when the expression level is at least 30% higher or lower than an expression level in a matched normal tissue.

Most typically, the genomics data comprises a whole genome sequencing data, a whole exome sequencing data or a copy number data, and/or the transcriptomics data comprises RNA sequencing data, RNA expression level data or allele fraction data. In some embodiments, the genomics data comprises sequencing data of a circulating tumor DNA and the transcriptomics data comprises a quantity of circulating tumor RNA. In some embodiments, the step of identifying the molecular signature comprises comparing a genome sequencing data of the tumor with a genome sequencing data of a matched normal tissue.

Preferably, the molecular signature comprises at least one of insertion or deletion of a nucleic acid fragment in a genome, wherein a size of the nucleic acid fragment is between 25-50 base pairs. Also preferably, the error-prone DNA repair is a non-homologous end joining (NHEJ) repair.

Various objects, features, aspects and advantages of the inventive subject matter will become more apparent from the following detailed description of preferred embodiments.

### DETAILED DESCRIPTION

The inventors have now discovered that a molecular signature of a tumor that is caused or affected by an error-prone DNA repair can be characterized with a short-length nucleotide insertion or deletion in the tumor genome, and further discovered that such molecular signature can be identified by analyzing tumor genomics and/or transcriptomics data. Such molecular signature can indicate or provide guidance for treatment of a tumor using a drug targeting the error-prone DNA repair pathway and/or a related mechanism that can be commonly used among the tumors sharing the molecular signature. Viewed from a different perspective, the inventors discovered that the effectiveness of a tumor treatment targeting the error-prone DNA repair pathway or a related mechanism can be determined or predicted by determining the presence of molecular signature related to the error-prone DNA repair pathway in the tumor.

Consequently, in one especially preferred aspect of the inventive subject matter, the inventors contemplate a method of analyzing omics data of a patient having a tumor. In this method, omics data sets of the tumor from the patient are obtained, and a molecular signature of an error-prone DNA repair in the tumor is identified from the omics data sets of the tumor. The inventors contemplate that such identified molecular signature can be associated with at least one of a causation, a prognosis, and/or a treatment option of the tumor.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

### Obtaining Omics Data Sets

Any suitable methods and/or procedures to obtain omics data or data sets are contemplated. For example, the omics data can be obtained by obtaining tissues from an individual and processing the tissue to obtain DNA, RNA, protein, or any other biological substances from the tissue to further analyze relevant information. In another example, the omics data can be obtained directly from a database that stores omics information of an individual.

Where the omics data is obtained from the tissue of an individual, any suitable method of for obtaining a tumor sample (tumor cells or tumor tissue) or normal (or healthy) tissue from the patient are contemplated. Most typically, a tumor sample or normal tissue sample can be obtained from the patient via a biopsy (including liquid biopsy, or obtained via tissue excision during a surgery or an independent biopsy procedure, etc.), which can be fresh or processed (e.g., frozen, etc.) until omics data of the tissue is available (e.g., obtained). For example, tissues or cells may be fresh or frozen. In another example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a normal tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of a cancer treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or normal tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

It is contemplated that tumor cells and/or some immune cells interacting or surrounding the tumor cells release cell-free DNA/RNA to the patient's bodily fluid, and thus may increase the quantity of the specific cell-free RNA in the patient's bodily fluid as compared to a healthy individual. As used herein, the patient's bodily fluid includes, but is not limited to, blood, serum, plasma, mucus, cerebrospinal fluid, ascites fluid, saliva, and urine of the patient. Alternatively, it should be noted that various other bodily fluids are also deemed appropriate so long as cell-free RNA is present in such fluids. The patient's bodily fluid may be fresh or preserved/frozen. Appropriate fluids include saliva, ascites fluid, spinal fluid, urine, etc., which may be fresh or preserved/frozen. Thus, in some embodiments, DNA (e.g., genomic DNA, extrachromosomal DNA, etc.), RNA (e.g., mRNA, miRNA, siRNA, shRNA, etc.) isolated and obtained from the patient's tumor sample can be cell-free DNA/RNA (cfDNA/RNA) or circulating tumor DNA/RNA (ctDNA/RNA).

Any suitable method for isolating cell-free DNA/RNA are contemplated. For example, in one exemplary method of DNA isolation, specimens were accepted as 10 ml of whole blood drawn into a test tube. Cell-free DNA can be isolated from other from mono-nucleosomal and di-nucleosomal complexes using magnetic beads that can separate out cell-free DNA at a size between 100-300 bps. For another example, in one exemplary method of RNA isolation, specimens were accepted as 10 ml of whole blood drawn into cell-free RNA BCT® tubes or cell-free DNA BCT® tubes containing RNA stabilizers, respectively. Advantageously, cell-free RNA is stable in whole blood in the cell-free RNA BCT tubes for seven days while cell-free RNA is stable in whole blood in the cell-free DNA BCT Tubes for fourteen days, allowing time for shipping of patient samples from world-wide locations without the degradation of cell-free RNA. Moreover, it is generally preferred that the cell-free RNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cell-free RNA in a collected tissue other than blood for at 2 days, more preferably at least 5 days, and most preferably at least 7 days. Of course, it should be recognized that numerous other collection modalities are also deemed appropriate, and that the cell-free RNA can be at least partially purified or adsorbed to a solid phase to so increase stability prior to further processing.

As will be readily appreciated, fractionation of plasma and extraction of cell-free DNA/RNA can be done in numerous manners. In one exemplary preferred aspect, whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). Cell-free RNA is extracted from 2 mL of plasma using Qiagen reagents. The extraction protocol was designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated cell-free RNA can be frozen prior to further processing.

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

Where it is desired to obtain the tumor-specific omics data, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, exemplary subtypes of genomics data may include, but not limited to genome amplification (as represented genomic copy number aberrations), somatic mutations (e.g., point mutation (e.g., nonsense mutation, missense mutation, etc.), deletion, insertion, etc.), genomic rearrangements (e.g., intrachromosomal rearrangement, extrachromosomal rearrangement, translocation, etc.), appearance and copy numbers of extrachromosomal genomes (e.g., double minute chromosome, etc.). In addition, genomic data may also include mutation burden that is measured by the number of mutations carried by the cells or appeared in the cells in the tissue in a predetermined period of time or within a relevant time period.

Moreover, it should be noted that some data sets are preferably reflective of a tumor and a matched normal sample of the same patient to so obtain patient and tumor specific information. In such embodiments, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

In addition, omics data of cancer and/or normal cells comprises transcriptome data set that includes sequence information and expression level (including expression profiling, copy number, or splice variant analysis) of RNA(s) (preferably cellular mRNAs) that is obtained from the patient, from the cancer tissue (diseased tissue) and/or matched normal tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

It should be appreciated that one or more desired nucleic acids or genes may be selected for a particular disease (e.g., cancer, etc.), disease stage, specific mutation, or even on the basis of personal mutational profiles or presence of expressed neoepitopes. Alternatively, where discovery or scanning for new mutations or changes in expression of a particular gene is desired, RNAseq is preferred to so cover at least part of a patient transcriptome. Moreover, it should be appreciated that analysis can be performed static or over a time course with repeated sampling to obtain a dynamic picture without the need for biopsy of the tumor or a metastasis.

Further, omics data of cancer and/or normal cells may comprise proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

Identification of Molecular Signature of an error-prone DNA repair

The inventors contemplate that the inaccurate or imperfect DNA repair by an error-prone DNA repair pathway, especially non-homologous end joining (NHEJ) pathway, are often accompanied with an insertion, deletion or a substitution of a relatively short-length nucleotide at the site of DNA double strand break. The length of nucleotides inserted, deleted, or substituted is generally longer than the random nucleotide mutations (e.g., single nucleotide insertion or deletion), and generally shorter than a chromosomal insertion (e.g, interchromosomal translocation, etc.). Preferably, the size of insertion, deletion or substitution resulted from the NHEJ is less than 200 base pairs, less than 150 base pairs, less than 100 base pairs, less than 50 base pairs, between 5-100 base pairs, between 10-100 base pairs, between 5-50 base pairs, between 10-50 base pairs, between 20-50 base pairs, or between 25-50 base pairs, etc. Thus, in a preferred embodiments, the molecular signature of the error-prone DNA repair in the tumor genome can be detected upon identifying an insertion, deletion, or substitution of less than 200 base pairs, less than 150 base pairs, less than 100 base pairs, less than 50 base pairs, between 5-100 base pairs, between 10-100 base pairs, between 5-50 base pairs, between 10-50 base pairs, between 20-50 base pairs, or between 25-50 base pairs, when compared the tumor genome sequence data (e.g., whole genome sequencing data, whole exome sequencing data, etc.) with the genome sequencing data of the matched normal tissue.

In some embodiments, the inventors contemplate that a pattern of the molecular signature can be determined from the omics data obtained from the tumor tissue. For example, where a plurality of omics data sets is obtained from a plurality of tumor samples from a patient at different time points (e.g., every 1 week, every 2 weeks, every 1 month, every 2 months, etc.), sequence comparisons among the tumor samples may show a cumulatively increased numbers of molecular signature of the error-prone DNA repair (e.g., insertion, deletion, substitution of short length nucleotide) over time that may indicate the ongoing or progressing malfunction of the DNA repair mechanism in the tumors. In another example, location and frequency of the molecular signature(s) in tumor genome (e.g., whether the molecular signatures are located more frequently in a specific chromosome or a locus of genome including a specific gene, etc.) can be identified to determine a pattern among tumor tissues among patients having similar conditions, prognosis, tumor types, or treatment histories.

Consequently, identified molecular signature(s) and/or their pattern(s) can be further associated with a (potential) causation and/or effect of the tumor. As used herein, the causation of the tumor refers to any direct or indirect molecular and/or cellular mechanism(s) (intrinsic or extrinsic) that can induce, develop, and/or maintain a tumor. The effect of the tumor refers to any direct or indirect molecular and/or cellular mechanism(s) and/or phenotype (e.g., physiological progress, shape, sensitivity, responsiveness, etc.) of the tumor that could be associated or preferably, causatively related to the molecular signature. In some embodiments, the causation may include an inactivity of core elements of NHEJ or DNA damage surveillance or response complex that may lead to error-prone DNA repair. In such embodiments, for example, the causation may include any loss or gain of activity in one of the core elements (e.g., Ku70/80, DNA-PKcs and XRCC4/LigIV) and/or one of the DNA damage surveillance or response complex (e.g., BRCA1, BRCA2, etc.) due to any mutation, change in RNA or protein expression levels due to transcriptional or translational regulations, and/or post-translational modifications (e.g., protein-protein binding, phosphorylati on, glycosylation, etc.).

The effect of the presence or accumulation of the molecular signatures may vary depending on the location (location in the chromosome, location in the gene, location in the exon, location in the intron, location in the promoter, location in the 5′- or 3′-untranslated region, location in the pseudogene, etc.), the length of the insertion/deletion/substitution, and/or the frequency of the insertion/deletion/substitution per chromosome, per gene, per given length of the chromosome (e.g., per 1 Mb, etc.). Exemplary effects may include a (hyper)mutation in a tumor-associated gene that may lead to a loss or gain of function of a protein encoded by the tumor-associated gene, expression of tumor-specific neoepitope, and/or loss of interaction between tumor cells and immune cells, etc. Thus, in some embodiments, the effect of the presence or accumulation of the molecular signatures can be determined from transcriptomics data of the tumor or the patient to determine RNA expression level of a gene having the molecular signature (a gene including the short-length insertion/deletion/substitution), optionally in association with RNA sequence data. For example, RNA expression level of the tumor-associated gene A having the molecular signature (e.g., an 25-base pair insertion in an exon) increases 50% compared to the matched normal, and such 25-base pair insertion results in translation of generation non-functional, dominant-negative protein encoded by the tumor-associated gene A, the effect can include overexpression of mutated gene, loss of function of the protein encoded by the mutated gene, and loss of function of a pathway associated with the protein encoded by the mutated gene due to the dominant negative function of the protein, etc.

In addition, identified molecular signature(s) and/or their pattern(s) can be further associated with a treatment option. For example, where the molecular signature(s) and/or their pattern(s) are present and/or detected from a tumor sample, such presence or detection can be associated with an undesirable treatment option including a DNA damaging reagent (e.g., alkylating, intercalating, strand breaking reagent, etc.) as such DNA damaging reagent may further accumulate the mutation in the tumor tissue due to the error-prone DNA repair pathway. Alternatively, such presence or detection can be associated with a treatment option including cell-death inducing reagent or immune therapy.

Pathway Analysis with Omics Data

Alternatively and/or additionally, the inventors contemplate that the causation and/or effect of the molecular signature of the error-prone DNA repair pathway can be identified and/or determined by pathway analysis. Thus, in some embodiments, one or more, preferably two or more types of omics data sets (e.g., genomics data, transcriptomics data, proteomics data, etc.) can be used to infer a pathway characteristic of the tumor tissue. From a different perspective, without wishing to be bound by any specific theory, the inventors contemplate that the mutational profiles, RNA expression profiles of the tumor tissue, and/or protein expression and activity data, either independently or collectively, affect the intracellular signaling networks, which consequently may change the intrinsic properties of the tumor tissues. Thus, so obtained omics data sets can be integrated into a pathway model to generate a modified pathway of tumor tissue to determine any differential pathway characteristic of the tumor tissue.

While any suitable methods of analyzing pathway characteristics of cells are contemplated, a preferred method uses PARADIGM (Pathway Recognition Algorithm using Data Integration on Genomic Models), which is a genomic analysis tool described in WO2011/139345 and WO2013/062505 and uses a probabilistic graphical model to integrate multiple genomic data types on curated pathway databases. In PARADIGM, a pathway model having a plurality of pathway elements (e.g., DNA sequence, RNA sequence, protein, protein function, etc.) can be accessed and a protein function or activity in the pathway can be inferred in a function of regulatory parameters in pathways using PARADIGM. Most typically, the pathway model comprises a plurality of pathway elements (e.g., proteins, etc.) that are connected by one or more regulatory nodes. For example, a pathway model [A] is a factor-graph-based pathway model (e.g., PARADIGM pathway model, etc.) that comprises pathway elements A, B, and C connected by a regulatory node I between the elements A and B, and another regulatory node II between the element B and C (A-I-B-II-C). The regulatory node I and II represent any factors other than A or B that may affect the activity of B and C. Thus, the pathway model [A] may be coupled to another pathway model [B] via one of the regulatory nodes I and II. Thus, in some embodiments, the pathway model may include a single pathway (e.g., NHEJ pathway, homologous recombination DNA repair pathway, etc.). Consequently, in some embodiments, the pathway model may be a single degree model that includes one or more signaling pathways that are parallel or substantially independent from each other. In other embodiments, the pathway model may be a multi-degree model that may include a plurality of signaling pathways that are coupled via one or more regulatory nodes (e.g., two degree model having pathways [A] and [B] where pathways [A] and [B] are coupled in a regulatory node of the pathway [A], three degree model having pathways [A], [B], and [C] where the pathways [A] and [B] are coupled in a regulatory node of the pathway [A] and pathways [B] and [C] are coupled in a regulatory node of the pathway [B].

The regulatory parameter(s) in each regulatory node may vary depending on the regulatory node connecting the pathway elements. For example, where the pathway element comprises a DNA sequence and the regulatory parameter is a transcription factor, a transcription activator, an RNA polymerase subunit, a cis-regulatory element, a trans-regulatory element, an acetylated histone, a methylated histone, and/or a repressor. Where the pathway element comprises an RNA sequence, the regulatory parameter is an initiation factor, a translation factor, an RNA binding protein, a ribosomal protein, an siRNA, and/or a polyA binding protein. Where the pathway element comprises a protein, the regulatory parameter is a phosphorylation, an acylation, a proteolytic cleavage, and an association with at least another protein.

Preferably, the inventors contemplate that the pathway analysis is performed with the pathways where at least one pathway element and/or a regulatory parameter is associated with molecular signature of error-prone DNA repair pathway. For example, the pathway model may include a DNA sequence of a gene that includes one or more short-length insertion/deletion/substitution as a molecular signature of the error-prone DNA repair. In another example, the pathway model may include a kinase encoded by a gene including one or more short-length insertion/deletion/substitution as a molecular signature of the error-prone DNA repair as a regulatory parameter of a pathway of protein B (that is phosphorylated by the kinase).

The pathway element activity of each pathway element can be inferred or calculated using the omics data as inputs in the central dogma module (DNA-RNA-protein-protein activity) as described in WO 2014/193982, which is incorporated by reference herein. For example, where the gene encoding protein A carries multiple genomic mutations in the exome, and RNA expression level of the gene increase upon a drug treatment, it can be inferred from such genomics and transcriptomics profile, the quantity of the protein may be increased while the activity of such protein may provide a dominant negative effect in the signaling pathway (where protein A is an element of the signaling pathway) due to missense mutations in the critical post-translational modification residues. Based on such inferred individual pathway element activity, the activity of downstream signaling pathway element can be inferred in the same signaling pathway or another signaling pathway that is connected by a regulatory node.

Consequently, diverse types of omics data can be integrated into a single pathway model to so allow on the basis of measured attributes (e.g., DNA copy number and/or mutations, RNA transcription level, protein quantities and/or activities) calculation of inferred attributes (e.g., DNA copy number and/or mutations, RNA transcription level, protein quantities and/or activities for which no data were obtained from the sample) and also calculation of inferred pathway activities. Advantageously, such calculations can employ the entirety of available omics data, or only use omics data that have significant deviations from corresponding normal values (e.g., due to copy number changes, over- or under-expression, loss of protein activity, etc.). Using such system, it should be appreciated that instead of analyzing only single or multiple markers, cell signaling activities and changes in such signaling pathways can be detected that would otherwise be unnoticed when considering only single or multiple markers in disregard of their function.

Preferably, the pathway models can be pre-trained via a machine learning algorithms (e.g., Linear kernel SVM, First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor) with omics data from the healthy individuals as inputs and corroborative data. In such embodiment, through the machine learning algorithms, each pathway element and the factor to the regulatory node will be provided with weights and directions to determine the activity of the downstream pathway elements. For example, where the pathway elements A and B are connected to regulatory node I, each, or at least one of quantity (e.g., copy number, expression level of RNA) and/or status (e.g., types and locations of mutations, number of phosphorylation for phosphorylated protein, etc.) of pathway element A and/or any factors of regulatory node I (e.g., activity of an enzyme affecting the activity of pathway element A, etc.) are integrated or calculated to infer the activity of pathway element B (e.g., quantity, status of protein B).

Consequently, such trained pathway model can be used as a template to predict how the pathway or pathway elements would be changed in the tumor tissue. For example, omics data obtained from the patient (and preferably compared with the matched normal tissue or normal tissue from healthy individuals) can be integrated into a factor-graph-based model using PARADIGM (or any suitable pathway models that can be machine-trained and produce reliable output data) to infer or predict which and how pathway elements would be changed due to the tumor-specific omics data changes compared to the compared with the matched normal tissue or normal tissue from healthy individuals. Thus, suitable pathway models include Gene Set Enrichment Analysis (GSEA, Broad Institute) based models, Signaling Pathway Impact Analysis (SPIA, Bioconductor) based models, and PathOlogist pathway models (NCBI) as well as factor-graph based models, and especially PARADIGM as described in WO2011/139345A2, WO2013/062505A1, and WO2014/059036, all incorporated by reference herein.

In some embodiments, a plurality of pathway models, each to infer an activity of a protein encoded by the DNA of the pathway, can be coupled to form one or more signaling pathway model of a tumor or a normal tissue. For example, where the signaling pathway includes pathway elements of protein A, protein B, and protein C, each pathway to infer the activity of protein A, B, or C, respectively (e.g., DNA-RNA-protein-protein activity pathway for protein A, protein B, protein C, respectively), can be coupled together to form a comprehensive signaling pathway. In some embodiments, the signaling pathway may include a DNA repair pathway (e.g., homologous recombination repair pathway, C-NHEJ, etc.), a cell cycle pathway, a cell proliferation pathway, an immune-stimulatory pathway (e.g., NK cell activation pathway, T cell activation pathway, etc.), an immune-inhibitory pathway (e.g., Treg activation pathway, etc.), immune-resistant pathway (e.g., tumor stem cell development pathway, immune evasion pathway, etc.). In other embodiments, the signaling pathway may include a metabolic pathway of a drug, a cell apoptosis pathway, a cell necrosis pathway, an inflammation pathway, and any other signaling pathways that are related to tumor initiation, development and prognosis and/or cell toxicity and death.

Additionally, such signaling pathway can be also characterized as a constitutively activated pathway, a functionally impaired pathway (e.g., due to reduced expression or reduced activity of a protein in the signaling pathway, etc.), and/or a dysregulated pathway (e.g., due to overexpressed, mutated protein that are dominant-negatively impact the signaling pathway, etc.), based on the inferred protein activity of one or more coupled pathway models.

In such embodiments, the pathway models can be modulated or modified in silico. For example, the pathway models can be modulated by adding the inferred activity of the tumor-associated protein (e.g., encoded by a gene having a molecular signature, etc.) as a variable (e.g., pathway element, etc.) or a regulatory parameter to simulate the pathway models to so infer how the pathway activity is changed and/or to infer how protein activity levels in one or more pathways are changed upon the tumor treatment in the tumor tissue and/or normal tissue. Optionally, the modulation of pathway models can be performed by adding a tumor treatment with which a patient has been treated with (optionally in different doses or different schedules as distinct variables or regulatory parameters) to simulate the pathway models to so infer how the pathway activity is changed and/or to infer how protein activity levels in one or more pathways are changed upon different doses or schedules of the treatment.

### Treatment Regimen and Prediction of Likelihood of Success

The inventors contemplate that the identified molecular signature of an error-prone DNA repair pathway, the associated causation and/or effect of the tumor with such identified molecular signature, and/or any intrinsic properties of a tumor determined by a pathway analysis associated with the molecular signature (i.e., as a pathway element or a regulatory parameter, etc.) can be further used to determine or generate a treatment regimen to treat the tumor and/or predict the response by the tumor tissue to a cancer treatment.

In some embodiments, it is contemplated that a treatment regimen or a treatment option can be generated or determined based on the at least one of the causation and the effect (e.g., prognosis) of the tumor associated with the molecular signature. For example, where the presence and/or accumulation of molecular signature in the genome in the tumor tissue is associated with an inactivity of DNA damage surveillance or response complex due to a mutation of BRCA1 or BRCA2, the treatment regimen may include a poly (ADP-ribose) polymerase (PARP) inhibitor treatment (e.g., Olaparib, etc.) that is known to be effective to treat the breast cancer associated with BRCA1 or 2 mutation(s). Without wishing to be bound by any specific theory, the inventors contemplate that where the two tumors share the same molecular mechanism (or molecular signature) associated with the causation of the initiation and/or development of tumor (e.g., BRCA1 mutation, failure of DNA damage surveillance or response, accumulation of mutations due to DNA damage and inaccurate DNA repair, etc.), the treatment known to (or predicted to) be effective to treat one tumor is likely to be effective to treat another tumor. Viewed from different perspective, the likelihood of success in treating a tumor with a PARP inhibitor is deemed high when the tumor carries the molecular signature of the error-prone DNA repair pathway.

In other embodiments, the treatment regimen can be determined or generated based on the inferred pathway activity using the molecular signature as begin associated with a pathway element. For example, where the presence and/or accumulation of molecular signature in the genome in the tumor tissue is associated with an overexpression of mutated protein (e.g., tumor-associated protein, etc., due to the insertion or deletion of short-length nucleotide in the coding region) or an overexpression of a protein (due to the insertion or deletion of short-length nucleotide in the promoter, enhancer, or any regulatory element in 5′-UTR) that can be attributed to the initiation, development, or maintenance of the tumor, the treatment regimen may include an inhibitor or an antagonist of the protein (e.g., an antibody, a binding motif, a binding protein, etc.), or an antagonist of mRNA from which the protein is encoded (e.g., siRNA, miRNA, etc.). For example, where the mutated protein is a kinase (with intact binding domain and mutated enzyme domain), the treatment regimen may include a kinase inhibitor, a pseudo-binding motif to the kinase, an antibody to the kinase, or an siRNA complementary to the mRNA encoding such mutated kinase.

In still other embodiments, the treatment regimen can be determined or generated based on the inferred pathway activity using the molecular signature as begin associated with a regulatory parameter or based on two inferred pathway activities. For example, where the pathway is a tumor-associated protein expression pathway (DNA->RNA->Protein->Protein activity), and the molecular signature (e.g., short insertion/deletion/substitution) of the error-prone DNA repair is present in a gene encoding a transcription factor that regulates the transcription of the gene encoding the tumor-associated protein, the activity of the regulatory parameter (a transcription factor having the molecular signature) can be inferred from another protein expression pathway of the regulatory parameter (a transcription factor having the molecular signature). Then, based on the inferred activity of the transcription factor as the regulatory parameter, the activity of the tumor-associated protein can be inferred. In such embodiments, the treatment regimen can be determined or generated to include an inhibitor or an antagonist of the tumor-associate protein (e.g., an antibody, a binding motif, a binding protein, etc.) or a treatment known to suppress the activity of the tumor-associate protein.

In another aspect of the inventive subject matter, the inventors further contemplate that the effectiveness of the cancer treatment can be determined or predicted based on the molecular signature and/or the expression of the gene having the molecular signature. For example, where the presence of a molecular signature of the error-prone DNA repair can be identified from omics data sets of a tumor tissue, the effectiveness of a PARP inhibitor to treat the tumor is predicted higher than to treat other tumors without the molecular signature. In another example, omics data sets can be obtained before, during, and after the tumor treatment with a PARP inhibitor and the presence and/or numbers of molecular signature in the omics sets can be compared. The effectiveness of the PARP inhibitor treatment can be determined “effective” where the omics data sets show absence and/or less numbers of molecular signatures in the tumor tissue (i.e., due to induced cell death of the tumor cells containing the molecular signature, etc.). Conversely, the effectiveness of the PARP inhibitor treatment can be determined “ineffective” where the omics data sets show increased or further accumulated numbers of molecular signatures in the tumor tissue. In such case, it is further contemplated that a second treatment regimen using different types of PARP inhibitor or different cancer treatment targeting directly the substrate of the error-prone DNA repair (e.g., a protein encoded by the gene containing the molecular signature, etc.).

In still another example, the effectiveness of the cancer treatment can be determined or predicted based on the molecular signature and the expression of the gene having the molecular signature. For example, where the presence of a molecular signature of the error-prone DNA repair is identified in gene A, and the expression of gene A is higher (e.g., at least 10%, at least 20%, at least 30%, at least 40%, at least 50%), or the expression of gene A is lower (e.g., at least 10%, at least 20%, at least 30%, at least 40%, at least 50%) than the matched normal, it is expected that the effectiveness of the PARP inhibitor would be high, as such treatment may prevent not only the error-prone DNA repair but also the abnormal cellular activity or intrinsic properties due to the over- or under-expressed gene A.

Preferably, such determined or generated treatment (regimen) can be further administered to the patient having the tumor in a dose and a schedule effective or sufficient to treat the tumor (e.g., to reduce the tumor size, to increase the immune response against the tumor, to increase the survival rate, etc.). In some embodiments, the dose and schedule can be determined to reduce the frequency or number of molecular signature(s) in the tumor tissue or patient's sample, or at least to suppress the accumulation of the molecular signature(s) in the tumor tissue or patient's sample. As used herein, the term “administering” refers to both direct and indirect administration of the treatment regimens, drugs, therapies contemplated herein, where direct administration is typically performed by a health care professional (e.g., physician, nurse, etc.), while indirect administration typically includes a step of providing or making the compounds and compositions available to the health care professional for direct administration.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of analyzing omics data of a patient having a tumor, comprising:
obtaining omics data sets of the tumor from the patient;
identifying a molecular signature of an error-prone DNA repair in the tumor from the omics data sets of the tumor; and
associating the molecular signature with at least one of a causation, a prognosis, and a treatment option of the tumor.

2. The method of claim 1, wherein the omics data sets includes at least two selected from genomics data, transcriptomics data, and proteomics data.

3. The method of claim 2, wherein the genomics data comprises a whole genome sequencing data, a whole exome sequencing data, or a copy number data.

4. The method of claim 1, wherein the transcriptomics data comprises RNA sequencing data, RNA expression level data or allele fraction data.

5. The method of claim 1, wherein the omics data sets include genomics data of a circulating tumor DNA or a transcriptomics data of a circulating tumor RNA.

6. The method of claim 1, wherein the molecular signature comprises at least one of insertion or deletion of a nucleic acid fragment in a genome, wherein a size of the nucleic acid fragment is between 25-50 base pairs.

7. The method of claim 1, wherein the error-prone DNA repair is a non-homologous end joining (NHEJ) repair.

8. The method of claim 1, wherein the identifying the molecular signature comprises comparing a genome sequencing data of the tumor with a genome sequencing data of a matched normal tissue, and/or wherein the causation comprises a mutation in at least one of BRCA1 and BRCA2.

9. (canceled)

10. The method of claim 1, further comprising determining an RNA expression level of a portion of a genome having the molecular signature, and optionally determining a treatment regimen to include a treatment targeting mRNA derived from the portion of the genome.

11. (canceled)

12. The method of claim 1, further comprising:
obtaining a pathway model comprising a plurality of pathway elements and a plurality of regulatory parameters;
inferring an activity of a tumor-associated protein using the pathway model and the omics data sets; and
wherein at least one of the pathway elements and the regulatory parameters includes the molecular signature of the error-prone DNA repair.

13. The method of claim 12, further comprising modulating the pathway model based on the inferred activity of the tumor-associated protein and/or determining a treatment regimen based on the at least one of the causation and the prognosis of the tumor.

14. (canceled)

15. The method of claim 13, wherein the causation is a mutation in at least one of BRCA1 and BRCA2, and the treatment regimen is a PARP inhibitor.

16. The method of claim 12, further comprising determining a treatment regimen to include a treatment targeting the tumor-associated protein.

17. A method of predicting effectiveness of a PARP inhibitor in treating a tumor of a patient, comprising:
obtaining genomics data and transcriptomics data of the tumor from the patient;
identifying a molecular signature of an error-prone DNA repair in the tumor from the genomics data of the tumor; and
determining an expression level of a portion of a genome having the molecular signature using the transcriptomics data; and
predicting the effectiveness of a PARP inhibitor based on the molecular signature and the expression level.

18. The method of claim 17, wherein the genomics data comprises a whole genome sequencing data, a whole exome sequencing data or a copy number data, and/or wherein the transcriptomics data comprises RNA sequencing data, RNA expression level data or allele fraction data.

19. (canceled)

20. The method of claim 17, wherein the genomics data comprises sequencing data of a circulating tumor DNA and the transcriptomics data comprises a quantity of circulating tumor RNA.

21. The method of claim 17, wherein the molecular signature comprises at least one of insertion or deletion of a nucleic acid fragment in a genome, wherein a size of the nucleic acid fragment is between 25-50 base pairs.

22. The method of claim 17, wherein the error-prone DNA repair is a non-homologous end joining (NHEJ) repair.

23. The method of claim 17, wherein the identifying the molecular signature comprises comparing a genome sequencing data of the tumor with a genome sequencing data of a matched normal tissue.

24. The method of claim 17, wherein the effectiveness of the PARP inhibitor is predicted high when the expression level is at least 30% higher or lower than an expression level in a matched normal tissue.
